(19) United States
(12) Patent Application Publication
(10) Pub. No.: US 2004/0146941 A1
(43) Pub. Date: Jul. 29, 2004

(54) Chemical encoding technology for combinatorial synthesis

(76) Inventors: Biliang Zhang, Shrewsbury, MA (US); Zhiyong Cui, Worcester, MA (US)

Correspondence Address: FISH & RICHARDSON PC, 225 FRANKLIN ST, BOSTON, MA, 02110, US

(21) Appl. No.: 10/698,932

(22) Filed: Nov. 3, 2003

Related U.S. Application Data
(60) Provisional application No. 60/423,619, filed on Nov. 4, 2002.

(51) Int. Cl. G01N 33/53; G01N 33/543

(52) U.S. Cl. 435/7.1; 436/518; 536/18.7; 546/334

(57) ABSTRACT

A chemical tag can include a core and a plurality of substituents attached directly to the core. The substituents of each chemical tag form a subset of a closed set of possible substituents. The tag can be used to track an object.

### CLAIM OF PRIORITY

This application claims priority to U.S. Patent Application Serial No. 60/423,619, filed on Nov. 4, 2002, the entire contents of which is hereby incorporated by reference.

### TECHNICAL FIELD

This invention relates to compounds and methods for use in combinatorial synthesis.

### BACKGROUND

The design, synthesis, and analysis of large chemical libraries has many important applications, for example in drug discovery and proteomics. Synthetic chemical libraries produced by combinatorial synthesis have rapidly become important tools for pharmaceutical lead discovery and compound optimization.

The determination of the chemical structure of biologically active library members is a major challenge. The quantity of material available from a large chemical library is frequently insufficient for conventional chemical analysis. One approach to determining the structure of library members is to associate the library members with tags that serve to record the reaction history of the library member.

### SUMMARY

A chemical tag can be used to encode the identity of an object, for example a solid support. In combinatorial or split-and-mix synthesis, one or more tags can be used to encode the reaction history and thus the identity of a compound linked to the solid support. The tags can be chemically inert so as not to interfere with synthesis of a compound linked to a solid support, or with a screen for biological activity of a compound linked to a solid support. The tags readily detected and readily distinguished from one another. The tags can each have a distinct mass, and the distinct mass can be the basis for distinguishing different tags.

In one aspect, in a family of chemical tags, each chemical tag includes a core and a plurality of substituents attached directly to the core, wherein the substituents of each chemical tag form a subset of a closed set of possible substituents.

In another aspect, in a plurality of different chemical tags each tag can include a core and a plurality of substituents attached to the core, at least one substituent including a repeating unit, and each different chemical tag including the repeating unit.

Each member of the family can include a different subset of substituents. The subset of substituents can include a repeating unit that is the same for all substituents of the subset. The core can be based on a polyhydroxy alkane. The core can be based on ethylene glycol, propylene glycol, glycerol, pentaerythritol, or a carbohydrate. Each chemical tag can include a charged or ionizable moiety. Each chemical tag can include a chromophore or fluorophore.

Each chemical tag can have the formula:

X—[Yᵢ—(R¹)ₘ—R²]ₙ

X can be a substituted or unsubstituted alkyl, cycloalkyl, heterocycloalkyl, alkoxy, acyl, alkenyl, cycloalkenyl, heterocycloalkenyl, alkynyl, aryl, aralkyl, or heteroaryl group.

Y can be, independently, selected from the group consisting of: —CR^{a}R^{b}—, —C(O)—, —S(O)—, —S(O)₂—, —O—, and —NR^{a}—, where each R^{a} and each R^{b} are independently hydrogen, halo, or a substituted or unsubstituted C₁-C₆ alkyl group.

Each i can be independently 1, 2, 3, 4, 5 or 6.

Each R¹ can be independently straight chain alkylene, branched chain alkylene, cycloalkylene, heterocycloalkylene, alkoxy, acyl, alkenylene, cycloalkenylene, heterocycloalkenylene, alkynylene, arylene, aralkylene, or heteroarylene, each R¹ independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group.

Each R² can be independently hydrogen or straight chain alkyl, branched chain alkyl, cycloalkyl, heterocycloalkyl, alkoxy, acyl, alkenyl, cycloalkenyl, heterocycloalkenyl, alkynyl, aryl, aralkyl, or heteroaryl, each R² independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group.

In the formula, n can be an integer ranging from 1 to 10.

Each m can be independently an integer ranging from 0 to 100.

In certain circumstances, each Y can be, independently, a group including one or more of the following moieties: —CH₂—, —C(O)—, —NR^{a}—, or —O—. In other circumstances, all R¹ are identical in at least one —Yᵢ—(R¹)ₘ—R² group. In other circumstances, each R¹ is identical in more than one —Yᵢ—(R¹)ₘ—R² group. N can be an integer ranging from 2 to 8; n can be 3, 4, 5 or 6. Each R¹ can be a straight chain alkyl group or a branched chain alkyl group. Each R² can be hydrogen. When each Y is —CH₂O—, X can be H₂N—CH₂—C—, and n can be 3. Each chemical tag can include a linker group. At least one chemical tag can be attached to a solid support through the linker group.

Each tag can have a mass distinguishable from the mass of other tags of the plurality. The core of each tag can be the same. Each tag can include a different number of repeating units. Each tag can have a different total m. Each tag can have a mass distinguishable from the mass of from other tags of the plurality.

In another aspect, a method of making a chemical tag includes selecting a subset of substituents from a closed set of possible substituents, and attaching each substituent of the subset directly to a core.

In another aspect, a method of making a family of chemical tags can include selecting a first subset of substituents and a second subset of substituents from a closed set of possible substituents, attaching each substituent of the first subset directly to a first core, and attaching each substituent of the second subset directly to a second core.

The subset can include at least two substituents. At least one substituent in the closed set of possible substituents can include a repeating unit. The method can include attaching a linker group to the core. The method can include attaching the tag to a solid support through the linker group. The first subset and the second subset can include different numbers of repeating units.

In another aspect, a method of tracking an object includes associating a chemical tag with an object, wherein the chemical tag includes a core and a plurality of substituents attached directly to the core, wherein the substituents of each chemical tag form a subset of a closed set of possible substituents, identifying the tag, and correlating the identity of the chemical tag with the object.

In another aspect, a method of tracking an object includes associating a plurality of different chemical tags with a plurality of objects, wherein each different chemical tag includes a core and a plurality of substituents attached directly to the core, at least one of the substituents including a repeating unit, each different tag including the repeating unit, determining the identity of an individual tag of the plurality of tags, and correlating the identity of the individual tag with an object of the plurality of objects.

Associating can include attaching the tag to the object. Identifying can include separating the tag from the object. Identifying can include determining a mass of the tag. Identifying can include determining a chromatographic retention time of the tag. The method can include associating a second chemical tag with the object. The method can include identifying the second chemical tag. The method can include chemically transforming the object before or after associating the chemical tag with the object. The object can include a support for solid phase synthesis. The support can be attached to a member of a library of compounds.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a pictorial representation of the split-and-pool method of combinatorial chemistry.

FIG. 2 shows the structures of 40 chemical tags.

FIG. 3 shows the mass spectra of ten tags.

FIG. 4 depicts the structures of the ten tags sampled in the MS and LC-MS analyses.

FIG. 5 shows the LC-MS chromatograms of the ten tags.

FIG. 6 shows a schematic diagram of encoding combinatorial synthesis and on-bead screening assay.

FIG. 7 shows structures for nine protected amino acid building blocks.

### DETAILED DESCRIPTION

One strategy for encoding combinatorial libraries is known as positional encoding or spatial encoding. Compounds are prepared by parallel synthesis, so that they remain physically separated from one another, for example in separate reaction vessels. In this approach, the location of the compounds allows their identification.

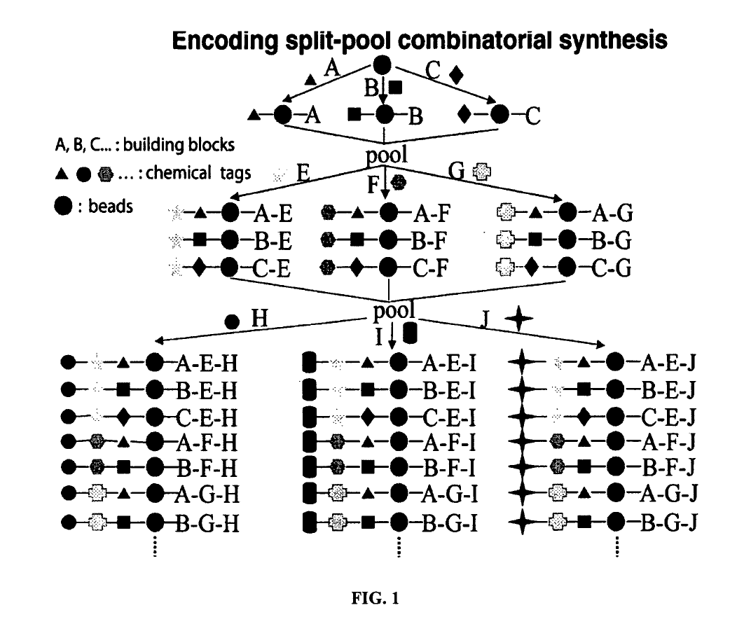

In another encoding strategy, the reactions are carried out on solid phase beads, with each bead having a different, specific compound bound to it. Each bead is labeled by chemical or physical identifiers or tags to allow the identification of the compound bound to the bead. Encoded beads can be mixed and assayed simultaneously. Encoded beads can be particularly useful for libraries prepared by split-and-pool synthesis (see FIG. 1).

Many of the approaches devised to prepare such libraries rely on solid-phase synthesis techniques and exploit the efficient split-and-pool or one-bead-one-compound method to assemble a statistical sampling of all possible combinations. The split-and-pool approach is gaining popularity within the field of combinatorial chemistry.

Encoding technology can provide opportunities to enhance the efficiency of the split-and-pool combinatorial approach. For larger libraries, an alternative encoding technique can be used to record the specific reaction history due to the larger library numbers.

According to accepted techniques of solid phase combinatorial synthesis, methods of attaching a tag (e.g., chemical or physical methods) to a bead allows identification of the sequence of synthetic steps in the synthesis of a specific compound. Multiple compounds are synthesized simultaneously on beads within the same reaction vessel by combining sets of preparative building blocks in just a few steps. The output of the split synthesis is a large number of compounds attached to the beads, each bead having one type of compound bound to the bead and each bead having thereto attached a tag to record the bead's unique reaction history.

In a specific example using the one-bead-one-compound strategy, a peptide library is generated by a solid phase technique using a split synthesis method. In split synthesis, the resin beads are divided into several aliquots of equal portions, and one each of 20 amino acids are added to each of 20 reaction vessels. The resins are then thoroughly mixed, deprotected and partitioned into 20 aliquots again for the next coupling cycle. The process is repeated several times until the desired peptide length is achieved. Since each resin bead encounters only one amino acid at each coupling cycle, and the reaction is driven to completion, the end result is that every peptide on each bead is unique. An enzyme-linked colorimetric assay can be used to screen the peptide bead library. Unlike the approach of using tags, the calorimetric approach solely provides an identification for “hits,” or positive reaction results, indicating that binding to the receptor has occurred. It did not provide a mechanism to determine the unique chemical identity of the specific ligands bound to the bead characterized as a “hit.” Advantageously, by having a unique identifier for the thousands of compounds that can be synthesized in libraries, unique chemical tags can be attached to entities such that hits in a chemical or biological assay can be identified by the tags.

To know which compound is bound to a particular bead, the coded bead can be identified by readily available analytical tools. The beads can be encoded during the library synthesis by adding a detectable chemical tag at each cycle that encodes for that particular step. In this strategy, which is termed chemical encoding, separation from the beads and chemical analysis of the tags is needed to identify the code, such as mass spectrometry or NMR.

Some encoding chemistries can interfere with the solid phase synthesis of compounds or with the assay identifying biological activity, resulting in artifacts. Therefore, alternative encoding strategies that overcome these limitations are desirable. Spectrometric encoding methods have been developed that make use of chemical tags.

The tag can include a core and a plurality of substituents attached directly to the core. The core can be derived from a polyhydroxy alkane, such as, for example, ethylene glycol, glycerol, pentaerythritol, or a carbohydrate. The polyhydroxy alkane can include other functional groups than hydroxy. The core can be a branching core, such that the substituents are all attached directly to the core.

The substituents can be selected from a closed set of possible substituents. When generating a family of tags from a set of possible substituents, no substituents are selected from outside the closed set. The substituents can include a repeating group. The closed set can be, for example, C₁-C₁₅ n-alkyl groups; in this example, a repeating group is —CH₂—. For each tag, a subset of substituents can be selected from the closed set of possible substituents. For example, if the closed set is C₁-C₁₅ n-alkyl groups, one subset of three substituents is C₂, C₂, and C₃; a different such subset is C₅, C₆, and C₇. A family of tags can be prepared, such that each member of the family includes a different subset of substituents from the closed set. The subsets can also be selected so that each member of the family has a different mass than any other member of the family.

The tag can include a linker group. The linker group can be attached to the core of the tag. The linker group can be attached to a solid support. A tag attached to a solid support through a linker group can be cleaved from the linker group. The tag can include a charged or ionzable moiety to facilitate detection by mass spectrometry. The charged or ionzable moiety can promote formation of positively charged species (e.g. an amine), or negatively charged species (e.g. a sulfonic acid).

The solid support can be used for solid phase synthesis. The tag can be used to encode the reaction history of a solid support. A set of different tags can be used to encode different reaction histories of individual solid supports. A tag can be attached to a solid support before or after the reaction that the tag encodes. The tags can be inert to the reaction conditions used for the solid phase synthesis. A compound made by solid phase synthesis can be unaalterted by the conditions used to attach or remove a tag from a solid support. A series of tags can each have a different mass. A series of tags can each have a different chromatographic retention time. The tag can include a chromophore or fluorophore to aid chromatographic detection, e.g. HPLC with on-line UV-vis or fluorescence detection. The tags can be detected by, for example, mass spectrometry (including LC-MS), HPLC, Capillary Electrophoresis-Mass Spectrometry (CE-MS), CE, and GC-MS.

The tags can be chemically inert and compatible with most chemical reaction conditions, such as oxidation, reduction, Michael additions, hydrogenations, Diels-Alder reactions, Suzuki coupling and other coupling reactions, acid and base conditions, Friedel-Crafts alkylation and acylation, and so on. Generally, a library of compounds encoded by the tags includes organic compounds. Synthesis of the library can involve the modification or introduction of one or more functionalities, ring openings, ring closings, expansions and contractions. The chemistry may further involve the use of nucleophiles, electrophiles, dienes, alkylating or acylating agents, nucleotides, amino acids, sugars, lipids, or variations thereof.

The tag can have the formula:

X—[Yᵢ—(R¹)ₘ—R²]_{n.}

X can be substituted or unsubstituted alkyl, cycloalkyl, heterocycloalkyl, alkoxy, acyl, alkenyl, cycloalkenyl, heterocycloalkenyl, alkynyl, aryl, aralkyl, or heteroaryl group.

Each Y can be, independently, selected from the group consisting of: —CR^{a}R^{b}—, —C(O)—, —S(O)—, —S(O)₂—, —O—, and —NR^{a}—, where each R^{a} and each R^{b} are independently hydrogen, halo, or a substituted or unsubstituted C₁-C₆ alkyl group.

Each i can be, independently, 1, 2, 3, 4, 5 or 6.

Each R¹ can be, independently, straight chain alkylene, branched chain alkylene, cycloalkylene, heterocycloalkylene, alkoxy, acyl, alkenylene, cycloalkenylene, heterocycloalkenylene, alkynylene, arylene, aralkylene, or heteroarylene, each R¹ independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group.

Each R² can be, independently, hydrogen or straight chain alkyl, branched chain alkyl, cycloalkyl, heterocycloalkyl, alkoxy, acyl, alkenyl, cycloalkenyl, heterocycloalkenyl, alkynyl, aryl, aralkyl, or heteroaryl. Each R², independently, can be optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group.

Each m can be, independently, an integer ranging from 0 to 100, and n can be an integer ranging from 1 to 10.

In certain circumstances, each R¹ is identical. Each R¹ can be a straight chain alkyl or branched chain alkyl group. Each R² can be hydrogen. Each Y can be —CH₂O—. When X is H₂N—CH₂—C—, n can be 3. When each R¹ is a straight chain alkyl or branched chain alkyl group, each m can be an integer ranging from 0 to 24. X can include a linker group that can attach to a solid support.

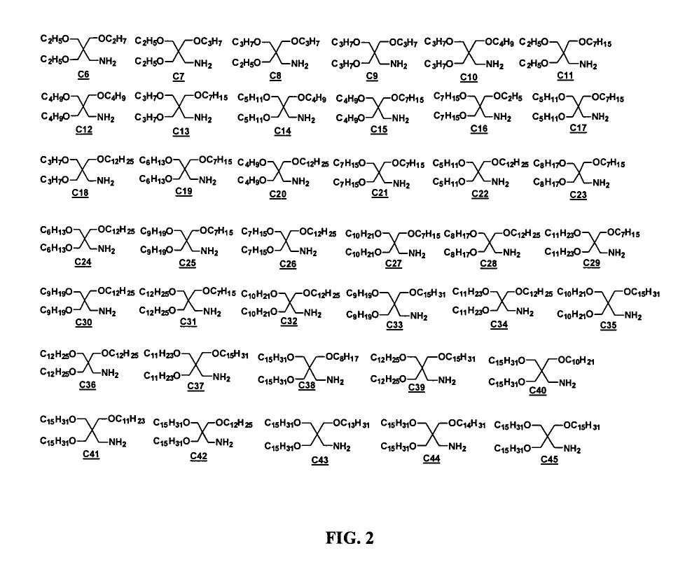

Compounds of the formula presented above can be prepared by ordinary synthetic organic chemistry. For example, a trialkoxypentaerythrityl amine (R3PEA) can be used as a chemical tag. The R3PEA tag can have the formula:

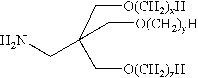

where x, y and z can each vary from 2 to 15. The structures of forty such tags are shown in FIG. 2. In FIG. 2, the tags are designated C2, C3, C4, . . . C45, according to the sum of x, y, and z. The tags can be prepared from pentaerythritol according to Scheme 1.

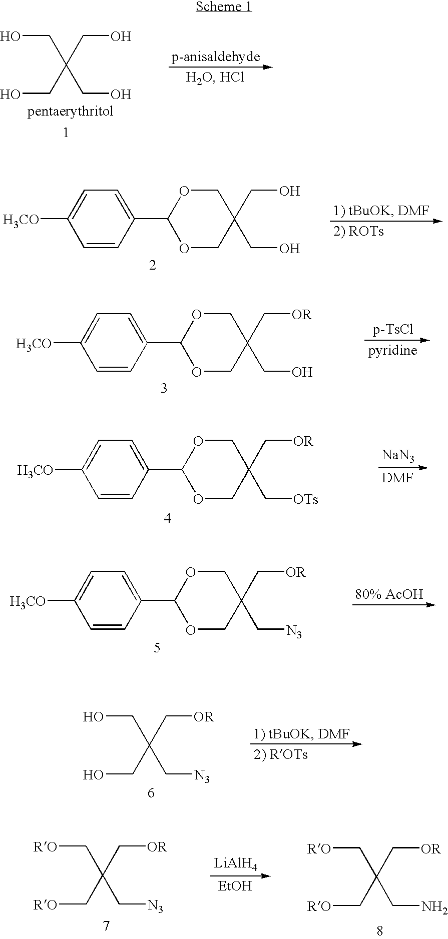

The tags can be modified to include a linker group, which can be attached to a solid support. For example, the linker group can include a tetramethyl benzyl alcohol. The preparation of a tag including such a linker is shown in Schemes 2 and 3.

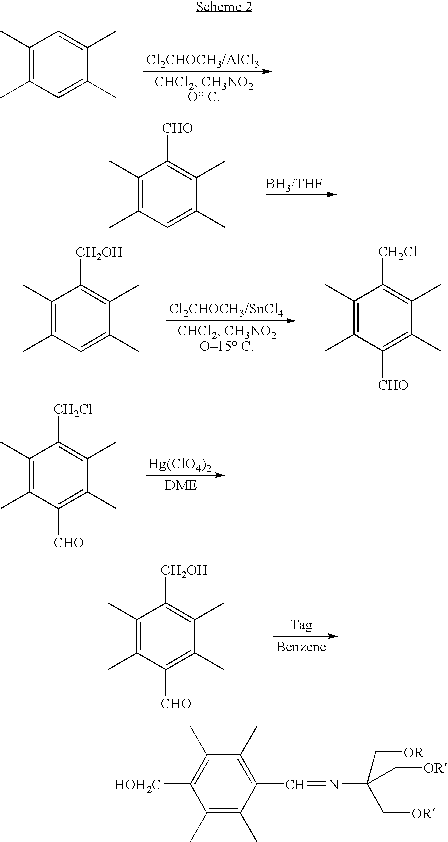

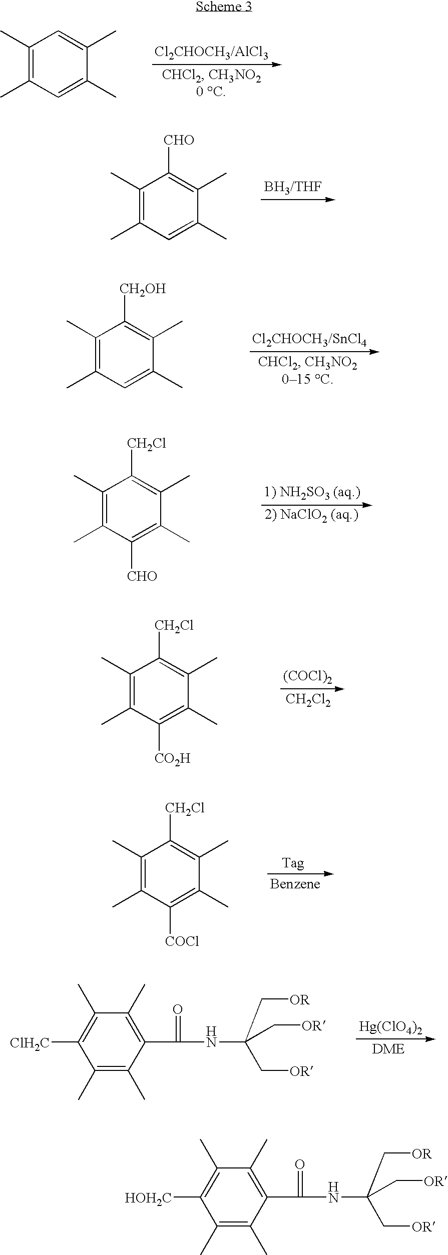

The benzyl alcohol group can be used to attach the tag to a solid support, for example, in the Friedel-Crafts alkylation of the aromatic rings of a polystyrene resin. Scandium(III) triflate and ytterbium(III) triflate catalyzes Friedel-Crafts alkylations to insert a set of hydroxyl pyrrole amide tags onto polystyrene resins (see Scott, R. H. et al. Chem. Commun., 1999, 1331, which is incorporated by reference in its entirety). Indium(III) triflate can be a more versatile catalyst to insert a hydroxymethyl benzyl amide R3PEA tag onto the polystyrene resins. See Scheme 4. A tag including a benzyl alcohol linker group can be attached to a polystyrene resin, a Wang resin, and a Rink resin.

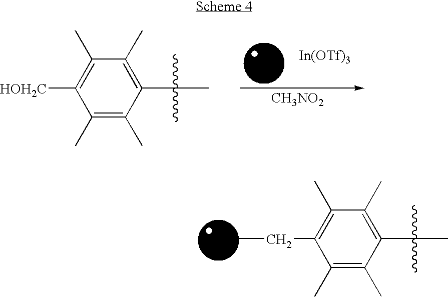

The tag can include a linker group that includes a diazoketone moiety, for example compound 17 in Scheme 5. A carbene generated from the diazoketone moiety can become linked to benzene (1°). In this way, a linker including a diazoketone can become linked to a phenyl group in a solid support, for example, a bead including polystyrene.

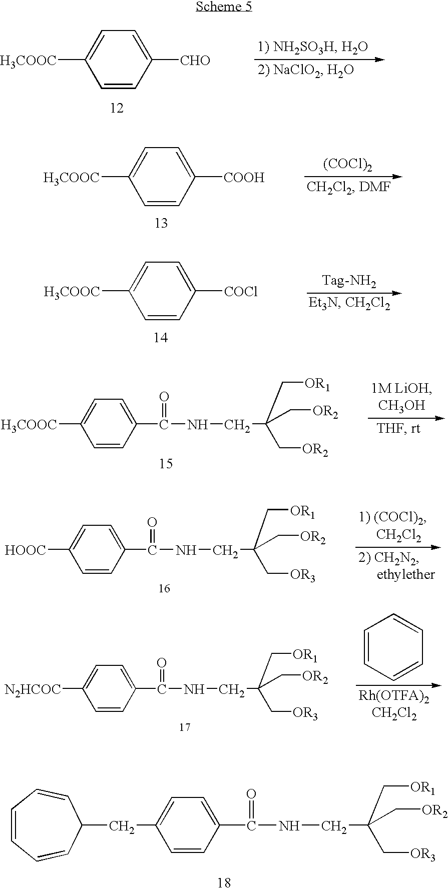

When attached to a solid support, the tags including linker groups of Scheme 2, 3 and 5, can be detached from the solid support under appropriate conditions. Specifically, the imine or amide linkages in these tags can be cleaved in acid at elevated temperature, for example 6N HCl at 150° C., 6N HCl at 130° C., 4 M HCl in dioxane, or HF in pyridine. In some cases it can be desirable to cleave a tag from a solid support under more mild conditions.

Scheme 6 shows a synthetic route to a tag inlcuding a linker group, 31, that includes an amide bond that can be cleaved in the presence of SnCl₂ in DMF at moderate temperature, such as 50° C. Compounds 30 and 31 in Scheme 6 are shown with three —C₁₅H₃, alkyl groups, though other R groups can be used.

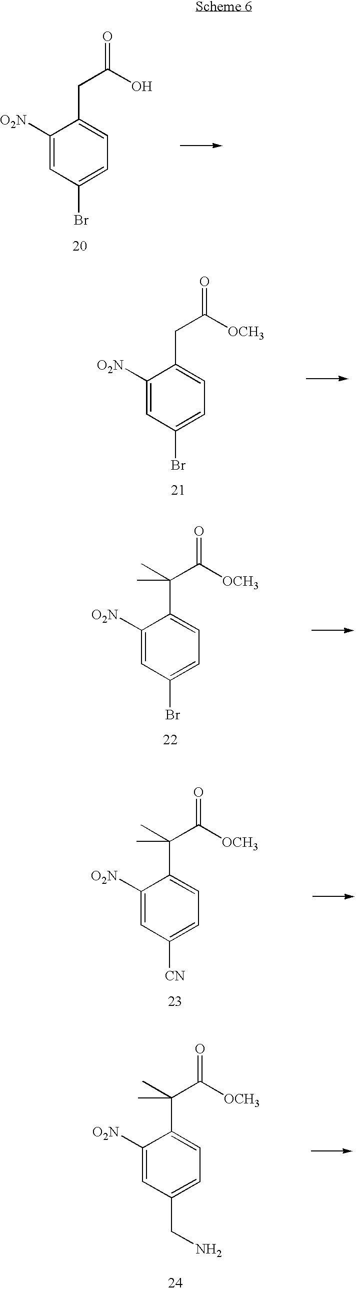

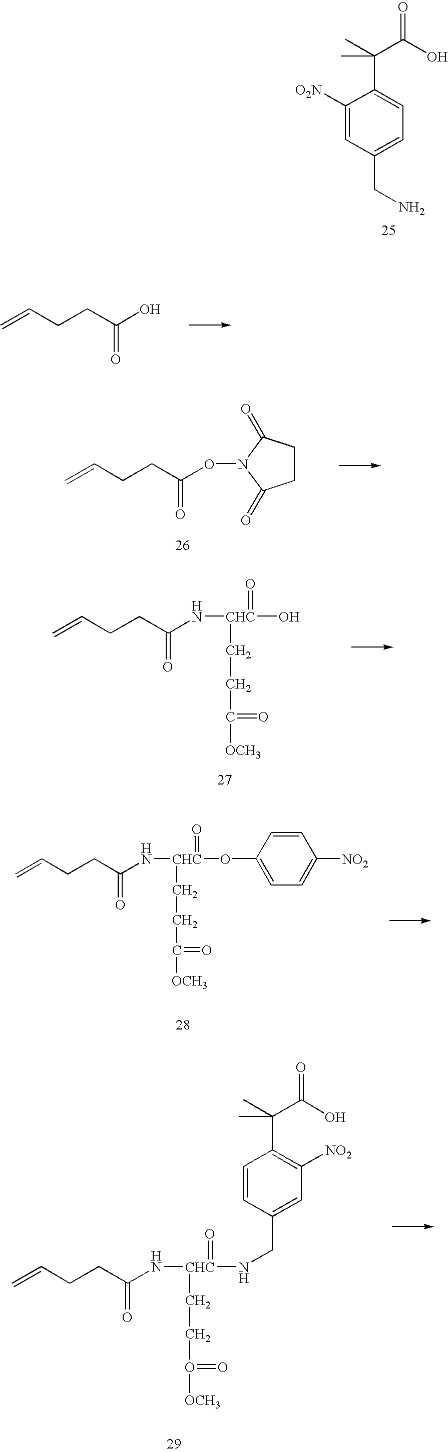

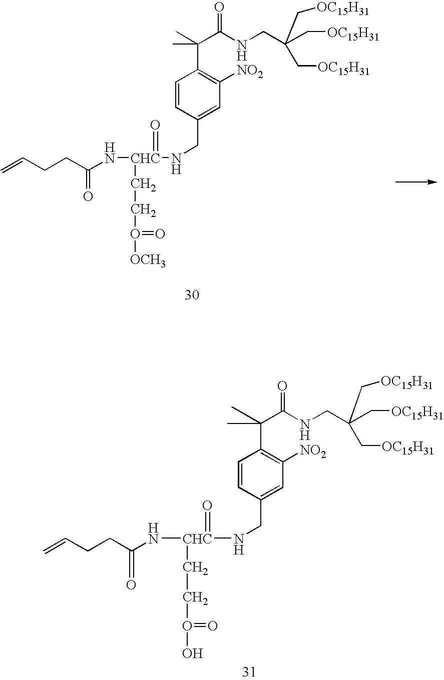

Alternatively, compound 23 can be prepared as shown in Scheme 7. The commercially available compound 43 was treated HCl, water and NaNO₂, then with NaCN and Cu(CN)₂ to give 44 which was nitrated to form compound 45. After the formation of methyl ester of 45 to give 46, 46 was methylated with CH₃I to give 23 with high yield.

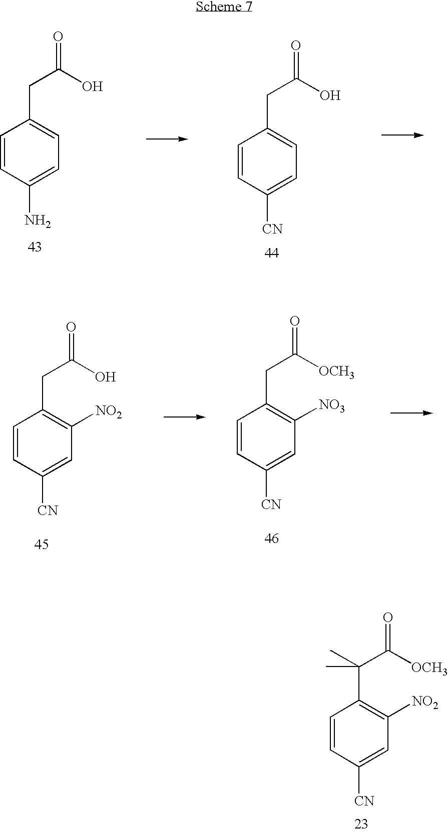

The tag including linker group 31 can be attached to a solid support that includes an amino group. Additional tags 31 can become attached to a tag that is attached to a solid support.

Compound 32 can be cleaved under very mild conditions (SnCl₂ in DMF at 50° C.).

Compound 32 was treated with 1.0 M tin chloride in DMF at 50° C. After 30 min., the tag was completely cleaved from the linker to form a ring closure product 33 and the tag 34 (Scheme 8). The majority of functional groups will be inert under these conditions.

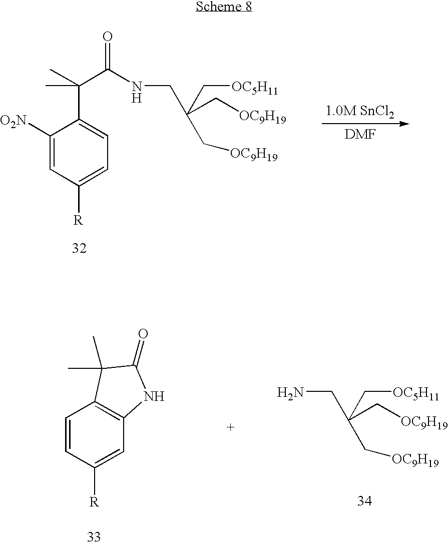

Commercially existing resin microbeads or macrobeads can be modified by attachment of a polyethylene glycol polymer chain for the encoding technique (Scheme 9). The amine-functionalized beads are reacted with a polyethylene glycol (PEG) with a an amine group protected by protecting group 1 (PG1) (e.g., 4-pentenoyl) in the short arm and a an amine group protected by a different protecting group (PG2) in the long arm. The chain can be characterized as having one long arm and, one short arm. The end of the long arm is designed to attach to the compound being synthesized, and the short arm is designed to be attached to tags. The beads serve as the solid support for combinatorial synthesis. Both reagents and tags anchor to the beads. The long and short arms can provide a more accurate synthesis and more efficient screening when compared to a typical bead modification due to the physical and chemical differentiation of the two arms. Because the two ends of the chain are designed to react with only tags or compounds, without cross-reaction, the appropriate chemicals will be in the appropriate places. Specifically, tags are confined to the short arm and the compounds to the long arm. Tags and compounds, once attached, cannot physically interact. This specificity ensures that the tags will not interfere with the compounds during on-bead screening.

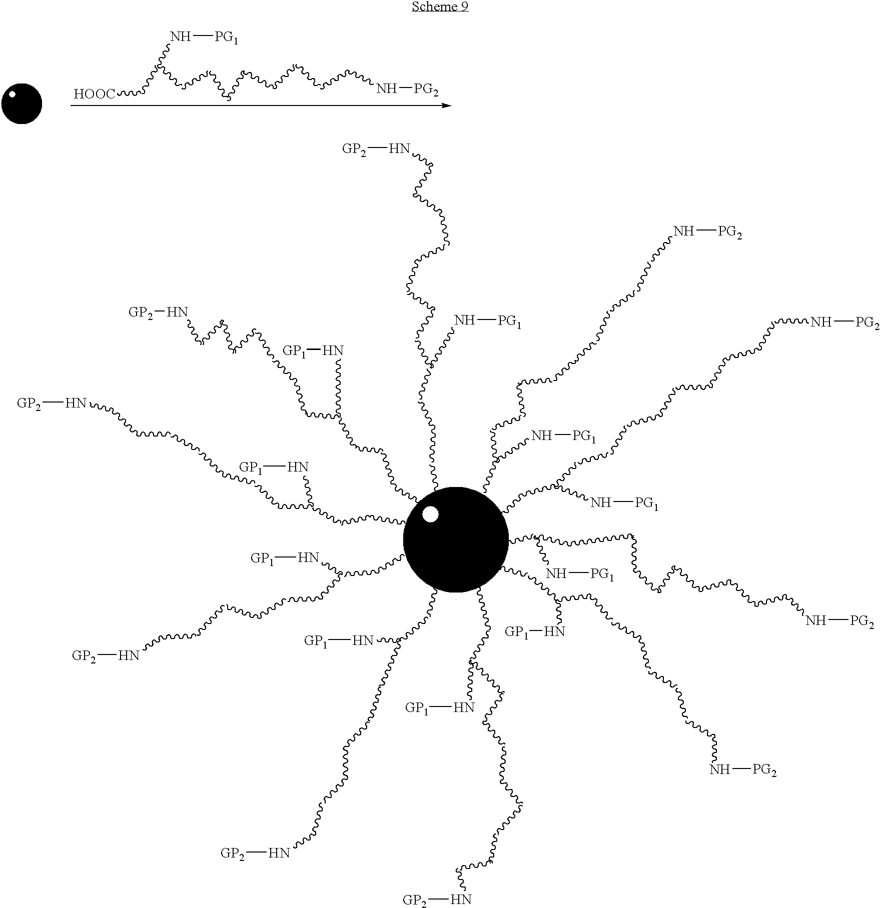

Scheme 10 shows the preparation of the polyethylene glycol modified beads, and the encoding strategy for tags like compound 31, Scheme 6. In Scheme 9, ‘NHS’ represents an N-hydroxysuccimide ester, ‘PYL’ represent the 4-pentenoyl protecting group, and ‘Block’ represents the sequentially added building blocks of a solid phase synthesis.

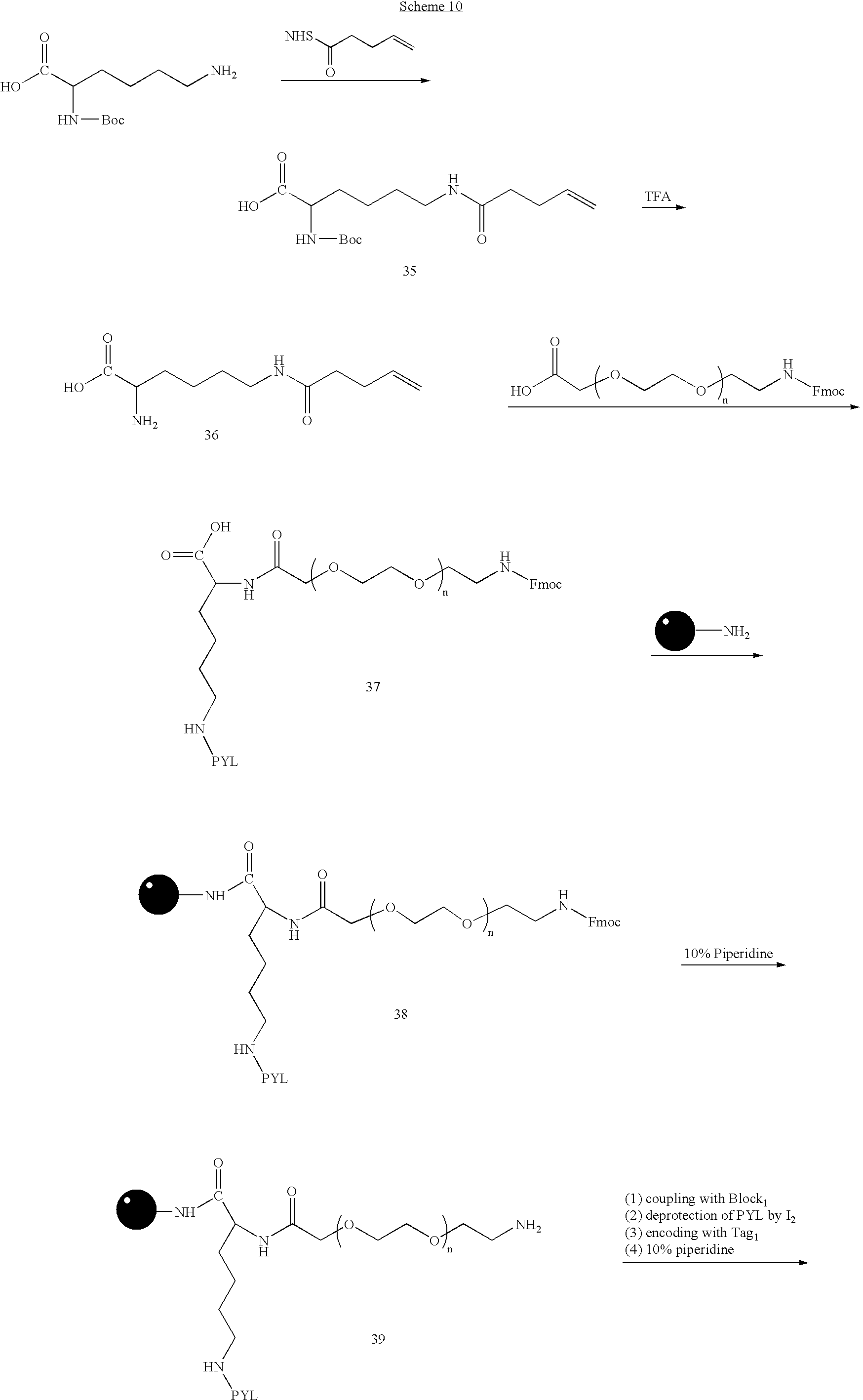

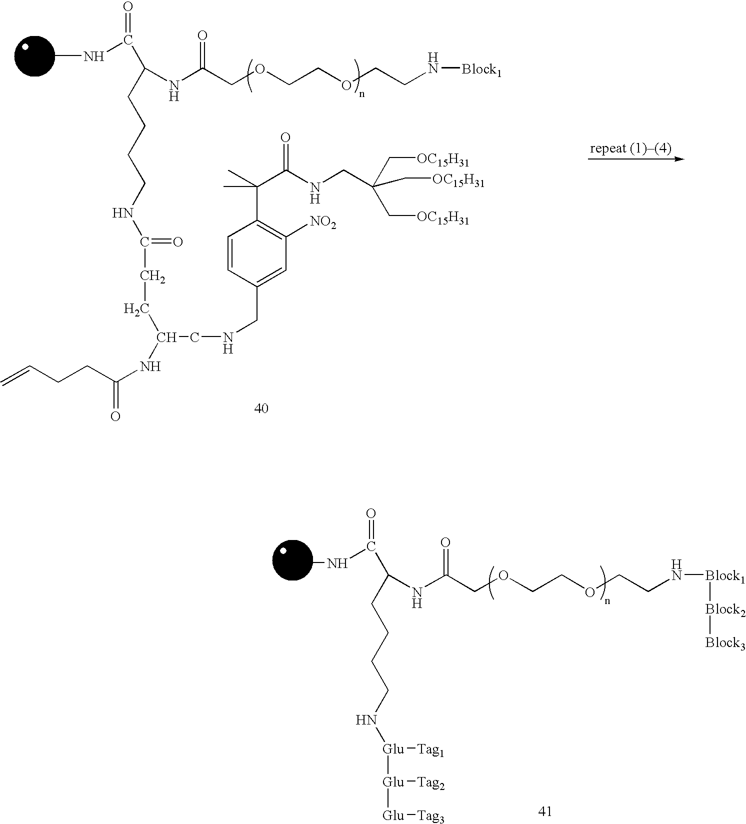

### EXAMPLE

By way of example, a tripeptide library was constructed by split-mix solid phase synthesis. The Rink resin was used as the solid phase and each step had three amino acid building blocks for three steps. Three batches of resins were coupled by means commonly known in the art. Essentially, this method used an Fmoc-amino acid building block using benzotriazol-1-ylotris(pyrrolidino)phosphonium hexafluorophosphate (PyBOP) chemistry for two hours. Other suitable coupling reagents may be used, such as bromo-tris-pyrrolidinophosphonium hexafluorophosphate (PyBrOP), HOAt/DIC, tetramethylfluoroformamidinium hexafluorophosphate (TFFH), or O-(7-azabenzotriazole)-N,N,N′,N′-tetramethyl-uronium-hexafluorophosphate (HATU). The resin was capped with acetic anhydride after each step of aminoacyl coupling and subsequently reacted the appropriate encoding hydroxymethyl benzyl amide R3PEA tag at 30-100 pmole per bead in 20 mM indium(III) triflate in a 1:4 solution of 1,2-dichloroethane:nitromethane for 5 hours. The beads were then mixed, split and the Fmoc group was completely deprotected for the next round of synthesis. The peptides were cleaved from a single bead in a sealed capillary tube with acid, preferably 98% TFA, and then the beads were subjected to acid hydrolysis. Preferably, the acid hydrolysis entails treatment of the beads with 6 N HCl at 135° C. in a sealed capillary tube to remove the R3PEA amine tag. The hydrolytic solution was then transferred to an eppendorf tube and the capillary tube was rinsed with acetonitrile and hexane. The solution was neutralized with sodium carbonate, extracted with hexane for three times and finally dried under Speedvac. The residue was dissolved in a 10 mM acetic acid-methanol solution, preferably in 2% heptane in 10 mM acetic acid-methanol, and confirmed by LC-MS analysis.

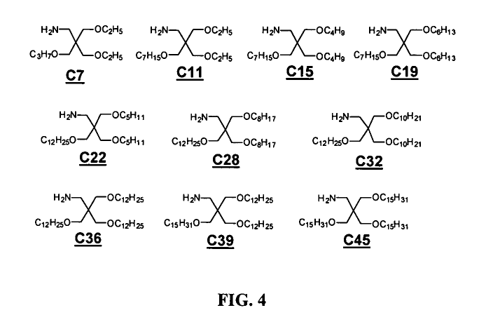

Ten different R3PEA tags were synthesized (C7, C11, C15, C19, C22, C28, C32, C36, C39, and C45; see FIG. 4) according to Scheme 1.

Synthesis of n-alkanol tosylates (serial code: TsOCₙH₂ₙ₊₁). General Procedure: To a mixture of the corresponding n-alkanol (CₙH₂ₙ₊₁OH, n=3-15; 0.86 mol; one eq.) and triethylamine (144 mL, 1.032 mol, 1.2 eq.) in dichloromethane (200 mL) was added a solution of tosyl chloride (188.7 g, 0.99 mol; 1.15 eq.) in dichloromethane (250 mL) in 15 min at 0° C. The solution was stirred at room temperature for 10-13 hours, to generate a light brown solution and white precipitate. After removing the precipitate (may have to filter two times) by filtration, 40 mL of ice-cold water and 100 mL of pyridine was added at 0° C. and the mixture was stirred at room temperature for 40-60 min until TsCl disappeared monitoring by TLC (ethyl acetate/hexane=1/5 and 1/1). After working up under standard manner, the oily residue was loaded onto a flash column of silica gel and eluted with hexane/diethyl ether to afford a colorless oil or white waxy solid. The yields are 90.8-93.0%.

Synthesis of Pentaerythritol Mono(p-Methoxybenzylidene Acetal) (2). The reaction of pentaerythritol 1 (90 g, 0.661 mol) with p-anisaldehyde was performed according to the classic method (C. H. Issidorides, R. Galen, Org. Synth. 1958, 38, 65-67.) in 85-88% yield. A suspension of pentaerythiol in 650 mL of water was stirred in a 80° C. water bath until a clear solution was obtained. The solution was cooled to room temperature, and 3.3 mL of concentrated HCl was added, followed by addition of 20 mL of p-anisaldehyde from an additional funnel. The addition of p-anisaldehyde took about 3 hours. After the addition was completed, the mixture was stirred for another 5 hours. The precipitate was collected by filtration and washed with ice-water solution and with a small amount of sodium carbonate (pH 8-9) for three times (3×150 mL) and then ice-water once. The solid was dried under vacuum overnight. The solid was washed again. The product was dried over vacuum and P₂O₅ in a dessicator overnight. A white solid was obtained. TLC: chloroform:methanol=9:1, R_{f}=0.43; chloroform:methanol=95:5, R_{f}=0.24; ethyl acetate:methanol=95:5, R_{f}=0.52; ethyl acetate: methanol=98:2, R_{f}=0.40. ¹H NMR (DMSO-d₆) 7.31 (d, J=8.8 Hz, 2H, 2 CH), 6.89 (d, J=8.8 Hz, 2H, 2 CH), 5.33 (s, 1H, CH(OCH₂)₂), 4.61 (t, J=5.2 Hz, 1H, CH₂OH), 4.52 (t, J=5.2 Hz, 1H, CH₂OH), 3.87 (d, J=12.0 Hz, 2H, CH₂O), 3.75 (d, J=12.0 Hz, 2H, CH₂O), 3.73 (s, 3H, OCH₃), 3.65 (d, J=5.2 Hz, 2H, CH₂OH), 3.22 (d, J=5.2 Hz, 2H, CH₂OH). ¹³C NMR (DMSO-d₆) 139.33, 131.22, 127.46, 113.24, 100.60, 69.03, 61.01, 59.52, 55.07.

Synthesis of 3. General Procedure: Using a three necked round-bottom flask equipped with mechanical stirrer, potassium tert-butoxide (24.7 g/150 mL in THF, 0.209 mol) was added to a solution of 2 (48.3 g, 0.19 mol) in anhydrous dimethylformamide (800 mL) in a dropwise manner for one hour with vigorous stirring. The mixture was stirred at room temperature for 0.5-1.0 hours to give a slurry solution. A solution of corresponding alkyl-tosylate (0.209 mol) in anhydrous dimethylformamide (200 mL) was then added dropwise to the above solution for 2 hours to afford a yellow clear solution. After stirring at room temperature for 8 hours, ice-cold water (250 mL) was added dropwise for 30 min until a precipitate just started to form. After working up under standard conditions, the crude product was purified two times by a flash column of silica gel eluted with hexane/ethyl acetate. The product was confirmed by NMR and MS.

Synthesis of 4. General Procedure: Tosyl chloride solid (228 mmol) was added to a solution of 3 (152 mmol) in pyridine (250 mL). The mixture was stirred at room temperature for 24 hours. The color of the reaction solution changed from green, to yellow, to orange and finally to light pink. After addition of cold water, the mixture was stirred for 0.5 hour and was evaporated to dryness. The residue was dissolved in diethyl ether (200 mL) and washed with water (3×200 mL) and brine (200 mL). The combined aqueous layers were extracted with diethyl ether (2×200 mL) and the combined organic layers were dried over anhydrous sodium sulfate and the solvent was removed in vacuo. The residue was loaded onto a flash column of silica gel and eluted with hexane/ethyl acetate (0-10%). Evaporation of the solvent under vacuum afforded a white solid in 84.6-90.0% yield.

Synthesis of 5. A mixture of corresponding 4 (100 mmol) and 2-3 equivalents of sodium azide (200-300 mmol) in anhydrous dimethylformamide (250 mL) was stirred at 130° C. for 20-24 hours. The reaction mixture was treated with water, extracted with methylene chloride (once) and washed with brine (200 mL). The organic layer was dried over anhydrous sodium sulfate. The solvent was removed in vacuo. The crude yellow solid was used directly in the next reaction without further purification.

Synthesis of 6. General Procedure: A mixture of above corresponding crude product (110 g) and 80% acetic acid (800 mL) was stirred at room temperature overnight resulting in a slight yellow solution. After removal of solvents under vacuum, the residue was dissolved in dichloromethane and stirred with activated carbon for a couple of hours and filtered through celite. The solvent was evaporated and the residue was loaded on a flash column of silica gel, eluted with dichloromethane and dichloromethane/methanol (0-2%) to give a colorless solid in 76.9-87.9% yield.

Synthesis of Azido Triether derivatives 7. General Procedure: To a solution of 6 (0.38 mmol) in anhydrous dimethylformamide (15 mL), was added potassium tert-butoxide (1 M solution in THF, 0.84 mL, 0.84 mmol). The mixture was stirred at room temperature for 4 hours to afford a yellow slurry. A solution of corresponding alkanyl tosylate (0.84 mmol) in anhydrous dimethylformamide (5 mL) was introduced through a transfer tube. After stirring at room temperature overnight (ca. 18 hours), an excess (0.2 mmol) of potassium tert-butoxide was added and the mixture was stirred for an additional 0.5 hour to decompose unreacted alkanol tosylate. After working up in the standard manner, the residue was loaded onto a flash column of silica gel and eluted with hexane/ethyl acetate to give the desired azido triethers 7 in 63.2-78.8% yield and small amount of azido diethers.

Synthesis of Amine Triether 8. General procedure: A mixture of corresponding azido triether 7 (0.27 mmol), ammonium formate (170 mg, 2.7 mmol), 10% Pd/C (30% w/w) and anhydrous methanol (6 mL) was stirred at room temperature for 6 hours (TLC indicated that the reaction after 2 days was similar to the reaction after only 6 hours). The mixture was filtered through celite and the solvent was removed in vacuo. The residue was dissolved in dichloromethane (100 mL), washed with water (2×20 mL) and brine (25 mL). The combined aqueous layers were extracted with dichloromethane (2×20 mL) and the combined organic layers were dried over anhydrous sodium sulfate. After evaporation to dryness, the residue was loaded onto a flash column of silica gel and eluted with a gradient of methanol (0-5%) in dichloromethane to give the desired product as a colorless oil or waxy solid in 72.7-82.8% yield. The products were confirmed by NMR and MS.

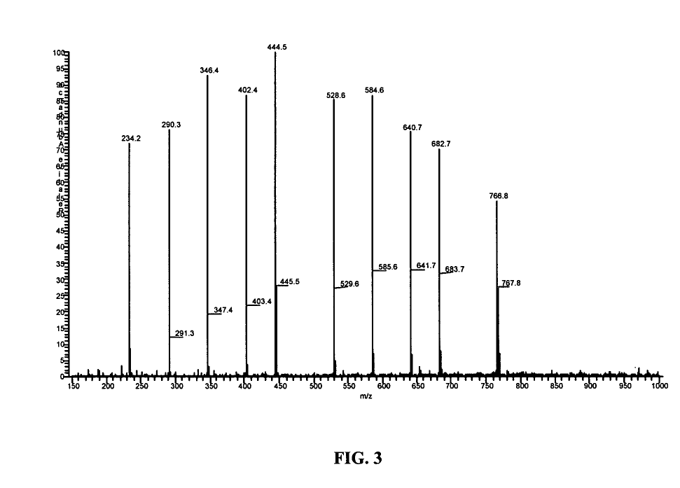

A stock solution having a 10 mM concentration of each of the 10 tags in nonane was diluted to 20 μM (each tag) with 10 mM HOAc in CH₃OH. Using a T connection, this 20 μM 10 tags stock solution was injected into a ESI-MS machine with a syringe pump at 2.5 μL per minute in an arm and a HPLC elutant with 90% CH₃OH (10 mM HOAc) and 10% of 10 mM HOAc was injected at 0.5 mL per minute in the other side simultaneously. The resulting MS spectrum was recorded (see FIG. 3). Meanwhile a tuning method was set up by tuning the molecular weight at 444.5. This method was saved as LC-MS tuning method.

ESI-MS Method Details:

Sheath gas flow rate 80

Auxiliary gas flow rate 35

Spray voltage 4.50 KV

Capillary temperature 270° C.

Capillary voltage 3 V

Tube lens offset 5V

Octupole 1 offset −3.75 V

Lens voltage −20.00 V

Octupole 2 offset −5.50 V

Octupole RF amplitude 400.00 V

LC-MS analyses were performed on the ThermoFinnigan LCQ^{DUO} system. TSP 4000 was used as the gradient pump, and the autosampler was an AS 3000. The detector was LCQ^{DUO} ESI-MS. The HPLC column was a Thermo Hypersil C18 reverse phase column (4.6×150 mm).

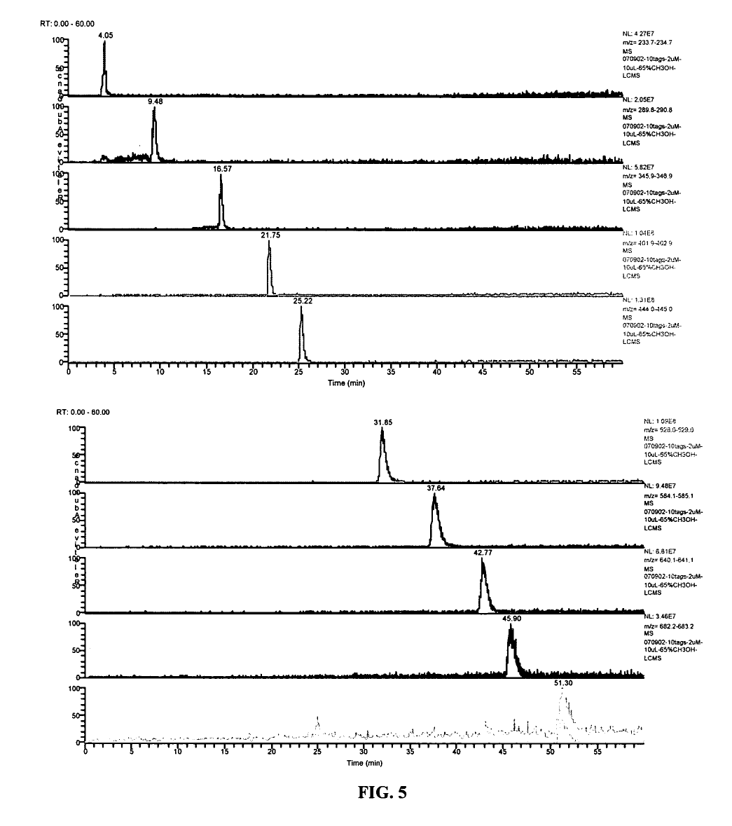

A ten-tag mixture (10 μL solution; stock solution in nonane diluted with CH₃OH (10 mM HOAc)) was injected by the AS 3000 autosampler into the LC-MS system in a 20 pmol concentration for each tag. HPLC elutants were A and B, with A consisting of CH₃OH (10 mM HOAc) and B consisting of 10 mM HOAc. The HPLC gradient program (0.5 mL per minute) started from 65% A and increasing to 90% A within 20 minutes, increasing from 90% to 98% of A within 20 minutes, from 98% to 100% of A within 10 minutes and keeping 100% A for 10 minutes. The ion signal was recorded by LCQ^{DUO}. The results of LC traces are depicted in FIG. 5. With almost five minutes between two adjacent peaks, it is likely that all forty tags could be separated with excellent resolution by LC-MS. A low loading (about 5 pmol tag) demonstrates the high sensitivity of the tags to LC-MS analyses.

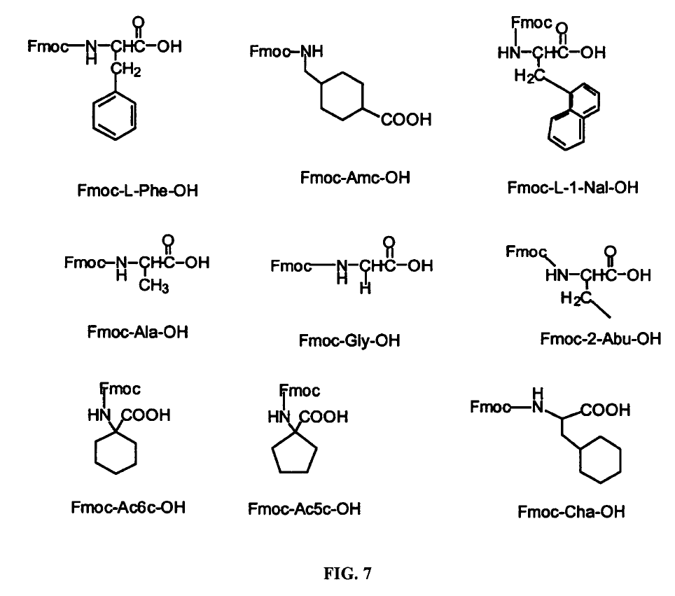

An encoded, 27-member tripeptide library was prepared by split-and-mix synthesis. Fmoc (9-fluorenylmethyloxycarbonyl) chemistry was used for the peptide synthesis. The solid support was beads of PL-Wang resin (Polymer Labs, 1.7 mmol/g, 200-250 EM). 20% piperidine in DMF (v/v) was used as the Fmoc deprotection reagent. Each amino acid was activated by PyBOP [Benzotriazol-1-yloxytris(pyrrolidino)phosphonium hexafluorophosphate] chemistry. The 9 amino acids used in the peptides (step 1: Gly, Phe, Ala; step 2: 2-Abu, Amc, Cha; step 3: Ac6c, Ac5c, 1-NaI) are shown in FIG. 7.

Resin (10 mg; 20 μmol loading capacity) was placed in each of 3 reaction vessels and was swelled with 10 μL of anhydrous DMF and 90 μL of methylene chloride for 60 min in 650 μL eppendorf tube. In the first step, diisopropylcarbodiimide (DIC) and HOBt were used as the coupling reagents. A solution of Fmoc amino acid (50 μmol) and HOBt (50 μmol) in 100 μL of DMF was added, then diisopropylcarbodiimide (20 pmol) and N,N-dimethylpyridine (DMAP, 2 μmol) were added. The suspension was rolled for 2 hours at room temperature. After the solvent was drained off, the resin was washed with DMF three times. This coupling reaction was repeated with fresh reagents. After the solvent was removed, the resin was washed with DMF three times. The resin was re-suspended in DMF and capped by Ac₂O (3.8 μL, 40 μmol) with rolling for 30 minutes. The DMF was removed and the resin was washed with CH₂Cl₂ three times. The resin was then suspended in CH₃NO₂ (1.0 mL) and reacted with the appropriate encoding tag (1.0 μmol, stock solution in ClCH₂CH₂Cl, approximately 11.5 nmol per bead, 5% relative to library loading) and 20 mM In(OTf)₃ or Sc(OTf)₃ for 2 hours with rolling at room temperature. The beads from three reaction vessels were then mixed first and then split into three reaction vessels in equal amounts. The Fmoc group was removed by a typical deprotection reagent. The next round of synthesis started. To a solution of Fmoc amino acid (50 μmol), HOBt (50 μmol) and PyBOP (50 μmol) in DMF (0.3 mL), DIEA (10.5 μL, 60 μmol) was added. The reaction solution was mixed thoroughly and was added to the N-deblocked resin immediately. The reaction mixtures were rolled for 2 hours. A total of three amino acid coupling steps were performed, giving a library of 27 different tripeptides.

Peptides from single beads were cleaved in a mixed reagent solution (TFA/Triisopropylsilane/Water, 95%/2.5%/2.5%) for 5 hours at room temperature. The supernatant was removed and analyzed by LC-MS. The beads were then sealed in a capillary tube and subjected to hydrolysis with H₂NNH₂ at 100° C. for 12 hr to detach the tags from the beads. The hydrolytic solution was extracted with chloroform three times. The combined organic layers were dried by Speedvac. The dried residue was subjected to LC-MS analysis.

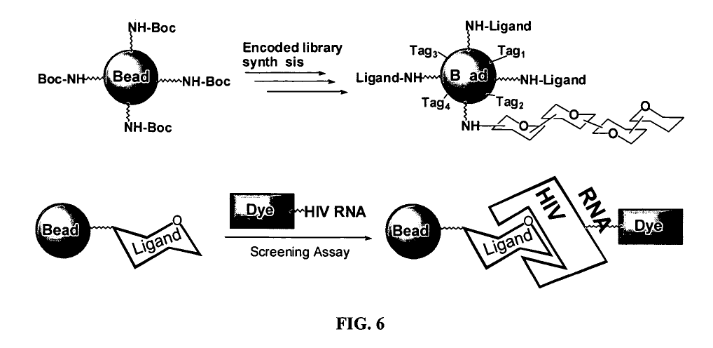

A pentapeptide mimic library is constructed to optimize the tag coupling conditions on a solid phase reaction as shown in FIG. 6. 12 tags are used for the binary encoding of 30 natural and/or unnatural amino acid building blocks listed in Table 1. The pentapeptide library is constructed by each step with 6 building blocks for 5 steps to form 7,776 compounds. The library can be screened against HIV RNA, ribosomal RNA and other virus RNA targets. An example of a screening assay is shown in FIG. 6. The RNA molecules are labeled with a fluorescence (e.g., red or green) tag at their 5′-end. The screening assay can be conducted with on-bead screening. For example, the active beads form complexes with the RNA target. The fluorescence-RNA of the complex can be detected under a microscope, or other means commonly used in the art. The active beads are then individually selected, and the tags cleaved from each bead, for example with 6 N HCl at 135° C. The tags are treated with sodium carbonate or other appropriate base, and then extracted with an organic solvent, such as heptane. The organic layers are then collected and dried over an appropriate drying agent, such as Na₂SO₄ or MgSO₄, and evaporated under vacuum. The final product is dissolved in 10 mM acetic acid in methanol and subjected to LC-MS analysis.

TABLE 1
Binary encoding of 30 natural or unnatural building blocks with 12 tags

| | C7 | C11 | C15 | C19 | | C21 | C23 | C25 | C29 | | C31 | C33 | C36 | C39 |
|---|---|---|---|---|---|---|---|---|---|---|---|---|---|---|
| aa1 | + | − | − | − | aa11 | + | − | − | − | aa21 | + | − | − | − |
| aa2 | − | + | − | − | aa12 | − | + | − | − | aa22 | − | + | − | − |
| aa3 | − | − | + | − | aa13 | − | − | + | − | aa23 | − | − | + | − |
| aa4 | − | − | − | + | aa14 | − | − | − | + | aa24 | − | − | − | + |
| aa5 | + | + | − | − | aa15 | + | + | − | − | aa25 | + | + | − | − |
| aa6 | + | − | + | − | aa16 | + | − | + | − | aa26 | + | − | + | − |
| aa7 | + | − | − | + | aa17 | + | − | − | + | aa27 | + | − | − | + |
| aa8 | − | + | + | − | aa18 | − | + | + | − | aa28 | − | + | + | − |
| aa9 | − | + | − | + | aa19 | − | + | − | + | aa29 | − | + | − | + |
| aa10 | − | − | + | + | aa20 | − | − | + | + | aa30 | − | − | + | + |

Thirty tags including cleavable linkers were prepared according to Scheme 6. Details of the synthesis are presented below.

4-Bromo-2-nitrophenylpyruvic acid methyl ester (21) To a solution of 20 (8.6 g, 33.21 mmol) in MeOH (160 mL) at ice-water bath, thionyl chloride (14.5 mL, 198.8 mmol) was added slowly. The reaction mixture was allowed to stir at room temperature for 2 hours. After the solvent was removed, the residue was dissolved in EtOAc (150 mL) and washed with water (100 mL) and saturated aqueous NaCl (100 mL). The organic layer was dried over anhydrous Na₂SO₄ and the solvent was removed under reduced pressure. The residue was purified by the column chromatography (SiO₂, 14-25% EtOAc in hexane) to give 21 (8.2 g, 90.4%) as white solid. The product was confirmed by NMR and MS spectrometer.

Methyl 2-(4-bromo-2-nitrophenyl)-2,2-dimethylacetate (22) To a solution of 21 (8.2 g, 30.04 mmol) and 18-crown-6 (0.794 g, 3 mmol) in anhydrous DMF (100 mL) cooled by ice-water bath was added iodomethane (7.5 mL, 120.20 mmol) under nitrogen atmosphere. The solution was stirred and sodium hydride (1.8 g, 75.1 mmol) was added in several portions within 1.5 hr. The reaction mixture was allowed warming gradually to room temperature and stirred over night. The solvent was removed under reduced pressure. Then the residue was suspended with CH₂Cl₂ (150 mL) and washed with water (50 mL). The organic layer was dried over anhydrous Na₂SO₄ and the solvent was concentrated under reduced pressure. The residue was purified by the column chromatography (SiO₂, 3-5% EtOAc in hexane) to give 22 (7.8 g, 86.3%) as yellow solid.

Methyl 2-(4-cyano-2-nitrophenyl)-2,2-dimethylacetate (23) A suspension of 22 (2 g, 6.64 mmol) and copper cyanide (12 g, 134 mmol) in anhydrous DMF (80 mL) was refluxed for 8 hr. The suspension was filtered through celite layer. Aqueous HCl (2 M, 35 mL) was added to the filtration. The mixture was extracted with ethyl ether (80 ml, twice). The organic layer was dried over anhydrous Na₂SO₄ and the solvent was concentrated under reduced pressure. The residue was purified by the column chromatography (SiO₂, 6-20% EtOAc in hexane) to give 23 (0.13 g, 7.9%) as yellow solid and starting material 22 (1 g).

Methyl 2-(4-methylamino-2-nitrophenyl)-2,2-dimethylacetate (24) Borane (2 mL, 1 M in THF) was added to 23 (0.15 g, 0.61 mmol) in a round bottom flask (50 ml). The reaction mixture was stirred at room temperature for 2 hr and was quenched by addition of several drops of HCl (6 M). The mixture was neutralized to pH=11 by NaOH (2.0 M). After the solvent was removed under reduced pressure, the residue was dissolved in CHCl₃ (50 mL) and washed with water (20 mL). The organic layer was dried over anhydrous Na₂SO₄ and concentrated under reduced pressure. The residue was run the column chromatography (SiO₂, 1-5% MeOH in CH₂Cl₂) to give 24 (0.1 g, 66.0%) as light yellow solid.

2-(4-Methylamino-2-nitrophenyl)-2,2-dimethylacetic acid (25) A solution of 24 (0.025 g, 0.099 mmol) in MeOH (2.5 mL) and NaOH (2.0 M) was refluxed for 4 hr. The mixture was neutralized to pH=1 with HCl (6.0 M). The precipitate was filtrated out and the filtration was applied to a reverse phase chromatography (1-50% MeOH in water) to give 25 (0.01 g, 42%) as yellow solid.

4-Pentenoic acid-N-hydroxysuccinimide ester (26). A mixture of 4-pentenoic acid (4.1 mL, 39.95 mmol), N-hydroxysuccinimide (5.1 g, 43.95 mmol), DMAP (0.54 g, 4.4 mmol) and DCC (9.07 g, 43.95 mmol) were dissolved in THF (200 mL) at 0° C. under nitrogen atmosphere. The reaction mixture was allowed warming to room temperature and stirred for 36 h. The reaction mixture was kept in freezer overnight. After the precipitate was filtered out, the solvent was removed under reduced pressure. The residue was purified by chromatography on a column of silica gel (0.02% CH₃OH in CH₂Cl₂) to give 26 (7.33 g, 93.1%) as white solid: R_{f}=0.50 (3.2% methanol in chloroform); ¹H NMR (400 MHz, CDCl₃) δ 2.47-2.52 (m, 2H), 2.70-2.74 (m, 2H), 2.84 (m, 2H), 5.07-5.16 (m, 2H), 5.82-5.89 (m, 1H); ¹³C NMR (100 MHz, CDCl₃) δ 25.81, 28.55, 30.52, 116.85, 135.38, 168.27, 169.34.

L-Glutamic acid-N-4-pentenoyl-5-methyl ester (27) To a suspension of 4-pentenoic acid-N-hydroxysuccinimide ester 26 (1.0 g, 5.1 mmol) and L-glutamic acid-5-methyl ester (0.9 g, 5.58 mmol) in anhydrous DMF (10 mL), diisopropylethylamine (3.54 mL, 20.32 mmol) was added slowly. The mixture was stirred at room temperature under nitrogen atmosphere for 26 hrs. The precipitate was filtered out and the filtration was condensed under the reduced pressure. The residue was dissolved in CH₂Cl₂ (200 mL) and washed with H₂O (30 mL). The organic layer was dried over anhydrous Na₂SO₄. After the solvent was evaporated under reduced pressure, the residue was purified by a column of silica gel (2-3% CH₃OH in CH₂Cl₂) to give 27 (0.61 g, 49.2%) as white solid.

4-Nitrophenyl N-4-pentenoyl-5-methyl ester-L-glutamate (28) To a solution of 27 (0.61 g, 2.51 mmol) and 4-nitrophenol (0.384 g, 2.76 mmol) in anhydrous THF (10 mL) cooled by an ice-water bath, dicyclohexylcarbodiimide (0.517 g, 2.51 mmol) was added. The mixture was allowed to stir at room temperature overnight. After the precipitate was filtrated out, the filtrate was concentrated to dryness. The residue was purified by a column of silica gel (5-50% EtOAc in hexane) to give 28 (0.91 g, 100%) as light yellow solid.

4-[(N-4′-pentenoyl 5-methyl ester-L-glutamatyl)-methylamino]-2-nitrophenyl-2,2-dimethylacetyl trialkoxypentaerythrityl amide (29) A mixture of 25 (0.24 g, 1.0 mmol) and 28 (0.44 g, 1.2 mmol) was dissolved in DMF (2 mL) and diisopropylethylamine (0.7 mL, 4.0 mmol) was added. The reaction mixture was stirred at room temperature under nitrogen atmosphere over night. After the solvent was removed, the residue was dissolved in CHCl₃ (50 mL) and was washed with water (20 mL). The organic phase was dried over Na₂SO₄. The solvent was removed and the residue was applied to column chromatography (1-20% MeOH in CH₂Cl₂) to give 29 (0.37 g, 80%) as white solid.

Fully protected linker-tag (30) A mixture of 29 (0.35 g, 0.755 mmol), N-hydroxysuccinimide (0.0956 g, 0.83 mmol), DMAP (0.009 g, 0.0755 mmol,) and DCC (0.171 g, 0.83 mmol) were dissolved in THF (20 mL) at 0° C. under nitrogen atmosphere. The reaction mixture was allowed to stir at room temperature over night. After the precipitate was filtered out, the solvent was removed under reduced pressure. The residue was chromatographed on a column of silica gel (0.02% CH₃OH in CH₂Cl₂) to give succinimide ester of 15 (0.38 g, 90%) as white solid.

To a solution of succinimide ester (0.38 g, 0.68 mmol) and C45-NH2 tag (0.52 g, 0.68 mmol) in THF (10 mL), diisopropylethylamine (0.47 mL, 2.71 mmol) was injected by syringe. The mixture was stirred at room temperature over night. The solvent was removed. The residue was dissolved in CHCl₃ (50 mL) and was washed with water (40 mL, twice). Then the organic phase was dried over Na₂SO₄. After the solvent was removed. The residue was applied to column chromatography (SiO₂, 5-50% EtOAc in hexane) to give 30 (0.97 g, 85%) as white solid.

Linker-Tag with free acid (31) A solution of 30 (0.90 g, 0.743 mmol) in THF (5 mL) was mixed a solution of lithium hydroxide monohydrate (5 mL, 1 M) in MeOH. The reaction mixture was stirred over night. Dilute HCl was dropped in very carefully to make weak acidic condition. Then the solvent was removed. The residue was dissolved in 50 mL of CHCl₃ and was washed with water (30 mL) and brine (30 mL). After the solvent was removed, the mixture was subjected to chromatography (SiO₂, 1-20% MeOH in CH₂Cl₂) to give 31 (0.8 g, 91.0%) as white solid.

γ-N-4-pentenoyl-Boc-lysine (35) To a suspension of 26 (1.0 g, 5.1 mmol) and α-Boc-lysine (5.61 mmol) in anhydrous DMF (10 mL), diisopropylethylamine (3.54 mL, 20.32 mmol) was injected. The mixture was stirred at room temperature under nitrogen atmosphere over night. The precipitate was filtered out and the filtration was condensed under reduced pressure. The residue was dissolved in CH₂Cl₂ (200 mL) and washed by H₂O (30 mL, twice). The organic layer was dried over Na₂SO₄. After the solvent was evaporated under reduced pressure, the residue was chromatographed on a column of silica gel (2-10% MeOH in CH₂Cl₂) to give 32 (88%) as white solid.

γ-N-4-pentenoyl-Boc-lysine-polyethylene glycol-ω-Fmoc-amine (37) To a solution of 35 (15 mmol) in DMF was added 10% piperidine in DMF for 1 hour. The reaction mixture was worked up under normal procedure and then used for next step. It was treated with co-Fmoc-amine-polyethylene glycol-COOH (10 mmol) in anhydrous DMF (10 mL) and diisopropylethylamine (3.54 mL, 20.32 mmol). The mixture was stirred at room temperature under nitrogen atmosphere overnight. The precipitate was filtered out and the filtration was condensed under reduced pressure. The residue was dissolved in CH₂Cl₂ (200 mL). The organic layer was dried over Na₂SO₄. After the solvent was evaporated under reduced pressure, the residue was chromatographed on a column of silica gel (2-10% MeOH in CH₂Cl₂) to give 37 (74%) as white solid.

γ-N-4-pentenoyl-Boc-lysine-polyethylene glycol-ω-Fmoc-amine modified Resin (38) Polystrene amino modified resin (1 g; 2.0 mmol loading capacity) was placed in a reaction vessel and swelled with 1.0 mL of anhydrous DMF and 1.5 mL of methylene chloride for 60 min. 37 (5 mmol, 10% of resin) and HOBt (0.2 mmol) in 0.5 mL of DMF was added, the diisopropylcarbodiimide (0.08 mmol) and N,N-dimethylpyridine (DMAP, 0.008 mmol) were added. The suspension was rolled for 2 hr at room temperature. After the solvent was drained off, the resin was washed with DMF three times. This coupling reaction was repeated with fresh reagents. The DMF was removed and the resin was washed with CH₂Cl₂ three times. The resins were dried over vacuum and ready for encoding library synthesis.

4-Cyano-phenylacetic acid (44) To a suspension of 4-amino-phenylacetic acid (18.2 g, 120.4 mmol), concentrated HCl (24.7 mL) and water (90 mL) warmed by a 40° C. water bath, acetic acid (13 mL) was added. This solution was cooled to 0-5° C. by an ice-water bath and a solution of sodium nitrite (9 g, 130.4 mmol) in water (32 mL) was added dropwise within 20 minutes. The orange solution was stirred for another 25 minutes at 0-5° C. and then it was added by a glass pipette (10 mL) slowly to a solution of sodium cyanide (29.5 g, 602 mmol), copper cyanide (21.6 g, 241 mmol) and water (280 mL) at 4-5° C. within 40 minutes. The black suspension was kept stirring at 4° C. for 1 hr and room temperature for 2 hr. The suspension was filtrated through celite and the precipitate was washed with EtOAc (50 mL, twice). The filtration was extracted with EtOAc three times. The combined organic layers were dried over anhydrous Na₂SO₄ and the solvent was concentrated under reduced pressure. The residue was applied to a column chromatography (SiO₂, 20% MeOH in EtOAc) to give 44 (15.3 g, 78.9%) as yellow solid.

2-Nitro-4-cyano-phenylacetic acid (45) To a solution of fuming nitric acid (60.7 mL) cooled by an ice-water bath, concentrated sulfuric acid (135.0 mL) was added and reaction temperature was controlled by the adding rate below 15° C. 44 (38.9 g, 241.4 mmol) was added in by several portions while the temperature of the mixture was between −9° C. and −4° C. After the mixture was stirred at 3-5° C. for another 2 hr., it was poured into the crushed ice (1500 g). The precipitate was filtrated out and washed by water to give 45 (45.8 g, 92%) as yellow solid after dried over vacuum.

Methyl 2-(4-cyano-2-nitrophenyl)-acetate (46) To a solution of 45 (45.8 g, 222.2 mmol) in MeOH (1100 mL) cooled by an ice-water bath, thionyl chloride (147 mL, 2015.2 mmol) was slowly added. The reaction mixture was allowed to stir at room temperature for 2 hours. After the solvent was removed, the residue was dissolved in EtOAc (400 mL) and was washed by water (100 mL) and saturated aqueous NaCl (100 mL). The organic layer was dried over anhydrous Na₂SO₄ and the solvent was removed under reduced pressure. The residue was purified by column chromatography (SiO₂, 17-25% EtOAc in hexane) to give 46 (42.6 g, 87.1%) as yellow solid.

Methyl 2-(4-cyano-2-nitrophenyl)-2,2-dimethylacetate (23) To a solution of 46 (14.4 g, 65.4 mmol) and 18-crown-6 (1.73 g, 6.54 mmol) in anhydrous DMF (100 mL) coolrf by an ice-water bath, iodomethane (16.4 mL, 263.4 mmol) was added dropwise. Then sodium hydride (3.92 g, 163.3 mmol) was added in several portions within 2 hr. The reaction mixture was allowed warming gradually to room temperature and stirred overnight. The solvent was removed under reduced pressure. Then the residue was suspended with CH₂Cl₂ (150 mL) and washed with water (50 mL). The organic layer was dried over anhydrous Na₂SO₄ and the solvent was concentrated under reduced pressure. The residue was applied to a column chromatography (SiO₂, 9-25% EtOAc in hexane) to give 23 (15.2 g, 93.7%) as yellow solid.

Fmoc (9-fluorenylmethyloxycarbonyl) chemistry is used to prepare an encoded tripeptide library. The reaction beads are PL-Wang amine resin (Polymer Labs, 1.7 mmol/g, 200-250 μM). 20% piperidine in DMF (v/v) is used as the Fmoc deprotection reagent. Each amino acid is activated by PyBOP [Benzotriazol-1-yloxytris(pyrrolidino)phosphonium hexafluorophosphate] chemistry. A split and mix 3 tripeptide library (step 1: Gly, Phe, Ala; step 2: 2-Abu, Amc, Cha; step 3: Ac6c, Ac5c, 1-NaI) is synthesized on PL-Wang amine resin. The structures of the 9 building blocks are shown in FIG. 7. The tags are of the type of compound 31 in Scheme 6.

Resin (10 mg; 18 μmol loading capacity) is placed in every reaction vessel (total of 3 vessels) and is swelled with 10 μL of anhydrous DMF and 90 μL of methylene chloride for 60 min in 650 μL eppendorf tube. A solution of one encoding block (5 μmol) and HOBt (5 mmol) in 10 μL DMF is added in one vessel, then diisopropylcarbodiimide (2 μmol) and N,N-dimethylpyridine (DMAP, 0.2 μmol) are added. The suspension is rolled for 2 hours at room temperature. Each vessel is treated with a different encoding block. After the solvent is drained off, the resin is washed with DMF three times. This encoding reaction is repeated with fresh reagents once again. After the solvent is removed, the resin is washed with DMF three times. The resin is re-suspended in DMF and capped by Ac₂O (3.8 μL, 40 μmol) with rolling for 30 minutes. The DMF is removed and the resin is washed with CH₂Cl₂ three times. A solution of Fmoc amino acid (50 μmol) and HOBt (50 μmol) in 100 μL of DMF is added, then diisopropylcarbodiimide (20 μmol) and N,N-dimethylpyridine (DMAP, 2 μmol) are added. A different building block is added to each reaction vessel. The suspension is rolled for 2 hours at room temperature. After the solvent is drained off, the resin is washed with DMF three times. This coupling reaction is repeated with fresh reagents. After the solvent is removed, the resin is washed with DMF three times. The resin is re-suspended in DMF and capped by Ac₂O (3.8 μL, 40 μmol) with rolling for 30 minutes. The DMF is removed and the resin is washed with CH₂Cl₂ three times.

To a suspension of the resin in 40 μL of 1:1 THF/H₂O (v/v) was added iodine (1.5 mg, 6 μmol). The reaction mixture is rolled at room temperature for 20 min, quenched with 0.5 M of Na₂S₂O₃ (24 μL, 12 μmol). After the solvent is drained off, the resin is washed with DMF and methylene chloride each for three times. The resin is dried over vacuum finally. The beads from three reaction vessels are then mixed first and then split into three reaction vessels in equal amounts. The next round of encoding starts with same method. Then the Fmoc group is removed by a typical deprotection reagent. The next round of library synthesis starts. To a solution of Fmoc amino acid (50 μmol), HOBt (50 μmol) and PyBOP (50 μmol) in DMF (0.3 mL), DIEA (10.5 μL, 60 μmol) are added. The reaction solution is mixed thoroughly and is added to the N-deblocked resin immediately. The reaction mixtures are rolled for 2 hours.

After three cycles, encoded peptide libraries are obtained. The peptides, from single beads, are cleaved in a mixed reagent solution (TFA/Triisopropylsilane/Water, 95%/2.5%/2.5%) for 5 hours at room temperature. The supernatant is removed and analyzed by LC-MS. The beads are then sealed in a capillary tube and subjected to reduction with tin (II) chloride at 50° C. for 2 hr. The hydrolytic solution is extracted with chloroform three times. The combined organic layers are dried by Speedvac. The residue is subjected to LC-MS analysis.

A number of embodiments have been described. Nevertheless, it will be understood that various modifications may be made. For example, the library is not limited to peptide libraries. Any other small molecule libraries can be synthesized by the encoding combinatorial synthesis. Accordingly, other embodiments are within the scope of the following claims.

What is claimed is:

1. A family of chemical tags, each chemical tag comprising a core and a plurality of substituents attached directly to the core, wherein the substituents of each chemical tag form a subset of a closed set of possible substituents.

2. The family of claim 1, wherein each member of the family includes a different subset of substituents.

3. The family of claim 1, wherein the subset of substituents includes a repeating unit that is the same for all substituents of the subset.

4. The family of claim 1, wherein the core is based on a polyhydroxy alkane.

5. The family of claim 4, wherein the polyhydroxy alkane is ethylene glycol, propylene glycol, glycerol, pentaerythritol, or a carbohydrate.

6. The family of claim 1, wherein each chemical tag includes a charged or ionizable moiety.

7. The family of claim 1, wherein each chemical tag includes a chromophore or fluorophore.

8. The family of claim 1, wherein each chemical tag has the formula:
X—[Yᵢ—(R¹)ₘ—R²]ₙ
wherein
X is a substituted or unsubstituted alkyl, cycloalkyl, heterocycloalkyl, alkoxy, acyl, alkenyl, cycloalkenyl, heterocycloalkenyl, alkynyl, aryl, aralkyl, or heteroaryl group;
each Y is, independently, selected from the group consisting of: —CR^{a}R^{b}—, —C(O)—, —S(O)—, —S(O)₂—, —O—, and —NR^{a}—, where each R^{a} and each R^{b} are, independently, hydrogen, halo, or a substituted or unsubstituted C₁-C₆ alkyl group;
each i is, independently, 1, 2, 3, 4, 5 or 6;
each R¹ is, independently, straight chain alkylene, branched chain alkylene, cycloalkylene, heterocycloalkylene, alkoxy, acyl, alkenylene, cycloalkenylene, heterocycloalkenylene, alkynylene, arylene, aralkylene, or heteroarylene, each R¹ independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
each R² is, independently, hydrogen or straight chain alkyl, branched chain alkyl, cycloalkyl, heterocycloalkyl, alkoxy, acyl, alkenyl, cycloalkenyl, heterocycloalkenyl, alkynyl, aryl, aralkyl, or heteroaryl, each R², independently, being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
n is an integer ranging from 1 to 10; and
each m is, independently, an integer ranging from 0 to 100.

9. The family of claim 8, wherein each Y is, independently, a group including one or more of the following moieties: —CH₂—, —C(O)—, —NR^{a}—, or —O—.

10. The family of claim 8, wherein all R¹ are identical in at least one —Yᵢ—(R¹)ₘ—R² group.

11. The family of claim 8, wherein each R¹ is identical in more than one —Yᵢ—(R¹)ₘ—R² m group.

12. The family of claim 8, wherein n is an integer ranging from 2 to 8.

13. The family of claim 8, wherein n is 3, 4, 5 or 6.

14. The family of claim 8, wherein each R¹ is a straight chain alkyl group or a branched chain alkyl group.

15. The family of claim 14, wherein each R² is hydrogen.

16. The family of claim 15, wherein each Y is —CH₂O—; X is H₂N—CH₂—C—; and n is 3.

17. The family of claim 8, wherein each chemical tag includes a linker group.

18. The family of claim 17, wherein at least one chemical tag is attached to a solid support through the linker group.

19. A plurality of different chemical tags each tag comprising a core and a plurality of substituents attached to the core, at least one substituent including a repeating unit, and each different chemical tag including the repeating unit.

20. The chemical tags of claim 19, wherein each tag has a mass distinguishable from the mass of other tags of the plurality.

21. The chemical tags of claim 19, wherein the core of each tag is the same.

22. The chemical tags of claim 19, wherein each tag includes a different number of repeating units.

23. The chemical tags of claim 19, wherein at least one tag has the formula:
X—[Yᵢ—(R¹)ₘ—R²]ₙ
wherein
X is a substituted or unsubstituted alkyl, cycloalkyl, heterocycloalkyl, alkoxy, acyl, alkenyl, cycloalkenyl, heterocycloalkenyl, alkynyl, aryl, aralkyl, or heteroaryl group;
each Y is, independently, selected from the group consisting of: —CR^{a}R^{b}—, —C(O)—, —S(O)—, —S(O)₂—, —O—, and —NR^{a}—, where each R^{a} and each R^{b} are, independently, hydrogen, halo, or a substituted or unsubstituted C₁-C₆ alkyl group;
each i is, independently, 1, 2, 3, 4, 5 or 6;
each R¹ is, independently, straight chain alkylene, branched chain alkylene, cycloalkylene, heterocycloalkylene, alkoxy, acyl, alkenylene, cycloalkenylene, heterocycloalkenylene, alkynylene, arylene, aralkylene, or heteroarylene, each R¹ independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
each R² is, independently, hydrogen or straight chain alkyl, branched chain alkyl, cycloalkyl, heterocycloalkyl, alkoxy, acyl, alkenyl, cycloalkenyl, heterocycloalkenyl, alkynyl, aryl, aralkyl, or heteroaryl, each R², independently, being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
n is an integer ranging from 1 to 10; and
each m is, independently, an integer ranging from 0 to 100.

24. The chemical tags of claim 23, wherein each tag has a different total m.

25. The chemical tags of claim 23, wherein each Y is, independently, a group including one or more of the following moieties: —CH₂—, —C(O)—, —NR^{a}—, or —O—.

26. The chemical tags of claim 23, wherein each R¹ is identical in at least one —Yᵢ—(R¹)ₘ—R² group.

27. The chemical tags of claim 23, wherein each R¹ is identical in more than one —Yᵢ—(R¹)ₘ—R² group.

28. The chemical tags of claim 23, wherein n is an integer ranging from 2 to 8.

29. The chemical tags of claim 23, wherein n is 3, 4, 5 or 6.

30. The chemical tags of claim 23, wherein each R¹ is a straight chain alkyl group or a branched chain alkyl group.

31. The chemical tags of claim 23, wherein each R¹ is —CH₂— and each R² is hydrogen.

32. The chemical tags of claim 23, wherein each tag has a mass distinguishable from the mass of from other tags of the plurality.

33. A method of making a chemical tag comprising:
selecting a subset of substituents from a closed set of possible substituents; and
attaching each substituent of the subset directly to a core.

34. The method of claim 33, wherein the subset includes at least two substituents.

35. The method of claim 33, wherein at least one substituent in the closed set of possible substituents includes a repeating unit.

36. The method of claim 33, further comprising attaching a linker group to the core.

37. The method of claim 36, further comprising attaching the tag to a solid support through the linker group.

38. A method of making a family of chemical tags, comprising:
selecting a first subset of substituents and a second subset of substituents from a closed set of possible substituents;
attaching each substituent of the first subset directly to a first core; and
attaching each substituent of the second subset directly to a second core.

39. The method of claim 38, wherein at least one substituent in the closed set of possible substituents includes a repeating unit.

40. The method of claim 39, wherein the first subset and the second subset include different numbers of repeating units.

41. A method of tracking an object comprising:
associating a chemical tag with an object, wherein the chemical tag includes a core and a plurality of substituents attached directly to the core, wherein the substituents of each chemical tag form a subset of a closed set of possible substituents;
identifying the tag; and
correlating the identity of the chemical tag with the object.

42. The method of claim 41, wherein associating includes attaching the tag to the object.

43. The method of claim 41, wherein identifying includes separating the tag from the object.

44. The method of claim 41, wherein identifying includes determining a mass of the tag.

45. The method of claim 41, wherein identifying includes determining a chromatographic retention time of the tag.

46. The method of claim 41, further comprising associating a second chemical tag with the object.

47. The method of claim 46, further comprising identifying the second chemical tag.

48. The method of claim 41, further comprising chemically transforming the object before or after associating the chemical tag with the object.

49. The method of claim 41, wherein the object includes a support for solid phase synthesis.

50. The method of claim 49, wherein the support is attached to a member of a library of compounds.

51. The method of claim 41, wherein the tag has the formula:
X—[Yᵢ—(R¹)ₘ—R²]ₙ
wherein
X is a substituted or unsubstituted alkyl, cycloalkyl, heterocycloalkyl, alkoxy, acyl, alkenyl, cycloalkenyl, heterocycloalkenyl, alkynyl, aryl, aralkyl, or heteroaryl group;
each Y is, independently, selected from the group consisting of: —CR^{a}R^{b}—, —C(O)—, —S(O)—, —S(O)₂—, —O—, and —NR^{a}—, where each R^{a} and each R^{b} are, independently, hydrogen, halo, or a substituted or unsubstituted C₁-C₆ alkyl group;
each i is, independently, 1, 2, 3, 4, 5 or 6;
each R¹ is, independently, straight chain alkylene, branched chain alkylene, cycloalkylene, heterocycloalkylene, alkoxy, acyl, alkenylene, cycloalkenylene, heterocycloalkenylene, alkynylene, arylene, aralkylene, or heteroarylene, each R¹ independently being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
each R² is, independently, hydrogen or straight chain alkyl, branched chain alkyl, cycloalkyl, heterocycloalkyl, alkoxy, acyl, alkenyl, cycloalkenyl, heterocycloalkenyl, alkynyl, aryl, aralkyl, or heteroaryl, each R², independently, being optionally substituted with one or more of an alkyl, cycloalkyl, alkenyl, cycloalkenyl, alkynyl, amino, alkylamino, acyl, alkoxy, hydroxyl, hydroxyalkyl, halo, haloalkyl, amino, aryl, or aralkyl group;
n is an integer ranging from 1 to 10; and
each m is, independently, an integer ranging from 0 to 100.

52. A method of tracking an object comprising:
associating a plurality of different chemical tags with a plurality of objects, wherein each different chemical tag includes a core and a plurality of substituents attached directly to the core, at least one of the substituents including a repeating unit, each different tag including the repeating unit;
determining the identity of an individual tag of the plurality of tags; and
correlating the identity of the individual tag with an object of the plurality of objects.

53. The method of claim 52, wherein associating includes attaching the plurality of different chemical tags to the object.

54. The method of claim 52, wherein identifying includes separating the plurality of different chemical tags from the object.

55. The method of claim 52, wherein identifying includes determining a mass of each of the different chemical tags.

56. The method of claim 52, wherein identifying includes determining a chromatographic retention time of the each of the different chemical tags.

57. The method of claim 52, wherein the object includes a support for solid phase synthesis.

58. The method of claim 57, wherein the support is attached to a member of a library of compounds.
